(19) United States
(12) Patent Application Publication
(10) Pub. No.: US 2010/0112660 A1
(43) Pub. Date: May 6, 2010

(54) Method for Derivatization of Proteins Using Hydrostatic Pressure

(75) Inventor: Mary S. Rosendahl, Broomfield, CO (US)

Correspondence Address: SHERIDAN ROSS PC, 1560 BROADWAY, SUITE 1200, DENVER, CO, 80202, US

(73) Assignee: BaroFold, Inc., Boulder, CO (US)

(21) Appl. No.: 12/475,234

(22) Filed: May 29, 2009

Related U.S. Application Data
(60) Provisional application No. 61/057,731, filed on May 30, 2008.

(51) Int. Cl. C07K 1/00; C07K 14/52; C07K 16/00; C12N 9/10; C12N 9/90

(52) U.S. Cl. 435/193; 530/402; 530/351; 530/391.1; 435/233

(57) ABSTRACT

The present invention provides an effective method for derivatization of proteins using hydrostatic pressure to reversibly perturb the native conformation of a protein such that a normally buried functional group on the protein, such as an amino acid residue, or a ligand or cofactor associated with the protein, is exposed and available for derivatization by a polymer molecule or a cytotoxic agent. The methods described herein do not require use of chaotropes, changes in pH, changes in temperature, or genetic modification of the native primary sequence of the protein and are applicable to substantially all proteins.

### CROSS-REFERENCE TO RELATED APPLICATIONS

This application claims the benefit of priority under 35 U.S.C. §119(e) from U.S. Provisional Application Ser. No. 61/057,731, filed May 30, 2008, the contents of which are incorporated herein in their entirety by this reference.

### FIELD OF THE INVENTION

The field of the present invention is protein biochemistry, in particular, derivatization of proteins to form biologically active polymer-protein or cytotoxic agent-protein conjugates.

### BACKGROUND OF THE INVENTION

Recombinant proteins frequently have a major drawback of a short circulating half-life in vivo which necessitates frequent administration of the protein and a higher risk of toxicity. One approach to improve the pharmacokinetic properties of a protein involves derivatization of the protein by attachment of a polymer molecule, such as polyethylene glycol (PEG), to the recombinant protein. [Harris, M. and Chess R B, Nat Rev Drug Discov. 2(3):214-21 (2003), Effect of pegylation on pharmaceuticals]

A commonly used method for derivatization involves linking the polymer molecule to free amines, such as at lysine residues or at the N-terminus. A major limitation of this approach is that proteins typically contain several lysine residues, in addition to the N-terminus. The polymer molecule attaches to the protein randomly at any of the available free amines, resulting in a heterogeneous product mixture. This heterogeneity is disadvantageous when developing a therapeutic derivatized protein product where predictability of biological activity is crucial.

A preferred approach is site-specific derivatization that results in a homogeneous preparation with the polymer molecule attached to a pre-determined location on the protein. Various approaches have been utilized that result in site-specific derivatization. Often it requires the protein to be genetically modified by the addition of a non-native amino acid, where the site specific derivatization takes place. However, such an approach may lead to a reduction in specific activity or the possibility of an immune response.

An alternative approach for site-specific derivatization involves attaching a polymer molecule to specific amino acid residues that are normally buried in the native conformation of the protein and thus are inaccessible to the bulky polymer molecules for chemical reactions. In such cases, partial denaturation of the protein molecule is necessary to expose the buried residue and to enable the derivatization reaction.

Typically, these protocols require the use of harsh chaotropes, detergents, solvents, and/or high temperatures to disrupt the native conformation. [Park, M. O., USPA 20050143563; El-Tayar et al., U.S. Pat. No. 6,638,500; Veronese et al., Bioconjugate Chem. (2007)] However, such an approach is likely to lead to irreversible aggregation and/or denaturation of the protein. Furthermore, the derivatization reactions are incomplete and particularly ineffective with reactive polymers that have molecular weights larger than 10 kDa. Additionally, a large excess of the polymer needs to be added, making this approach impractical for a recombinant protein with therapeutic potential.

A second approach that is used to derivatize a buried amino acid residue in a recombinant protein uses a two step reaction. [Bossard, M., US Pat. Appl. Pub. 20070092482; El-Tayar et al., U.S. Pat. No. 6,638,500] The protein is first treated with a small heterolinker reagent that is subsequently coupled to a larger polymer molecule. Because of limited reactivity, the protocol requires using a 40 to 50 fold excess of the small PEG polymer and a 20 to 100 fold excess of the larger PEG polymer, and requires application of extra purification steps. Two step methods are likely to result in a heterogeneous mixture of polymer-protein conjugate variants.

Improved methods for derivatizing proteins that overcome the limitations of the current technology are needed. Specifically, methods that allow for the production of derivatized proteins without significant heterogeneity and reductions in specific activity are desirable. In addition, efficient methods of production of such derivatized proteins having reduced or no risk of generating an immune response would be an advance in the art.

### SUMMARY OF THE INVENTION

In one embodiment, the instant invention comprises a method for derivatization of a protein comprising the steps of applying hydrostatic pressure to the protein to increase reactivity of a functional group on the protein, contacting the functional group on the protein with a reactive polymer molecule to form a polymer-protein conjugate, and depressurizing the polymer-protein conjugate.

In another embodiment, the instant invention comprises a method for derivatization of a protein comprising the steps of applying to the protein a hydrostatic pressure of about 0.1 to about 25 kilobars, contacting a functional group on the protein with a reactive polymer molecule to form a polymer-protein conjugate, and depressurizing the protein-polymer conjugate.

In another embodiment, the instant invention comprises a method for derivatization of a protein comprising the steps of applying hydrostatic pressure to the protein to increase reactivity of a functional group on the protein, contacting the functional group on the protein with a cytotoxic agent to form a cytotoxic agent-protein conjugate, and depressurizing the cytotoxic agent-protein conjugate.

In another embodiment, the instant invention comprises a method for derivatization of a protein comprising the steps of applying to the protein a hydrostatic pressure of about 0.1 to about 25 kilobars, contacting a functional group on the protein with a cytotoxic agent to form a cytotoxic agent-protein conjugate, and depressurizing the cytotoxic agent-protein conjugate.

In some embodiments, the step of applying hydrostatic pressure to the protein comprises applying a hydrostatic pressure of about 0.25 to about 5 kilobars, or about 1 to about 3 kilobars. In some embodiments, the hydrostatic pressure applied to the protein is sufficient to alter native conformation of the protein.

In some embodiments, the instant invention comprises the step of recovering the polymer-protein conjugate or the cytotoxic agent-protein agent conjugate.

In some embodiments, the molar ratio of the reactive polymer molecule or the cytotoxic agent to the protein is less than about 20, less than about 10, or less than about 5. In preferred embodiments the reactive polymer molecule is PEG.

In some embodiments, after depressurization the derivatized protein is brought to atmospheric pressure.

In some embodiments, the reactive polymer molecule or the cytotoxic agent is present with the protein during the step of applying hydrostatic pressure to the protein.

In one embodiment, the instant invention comprises a composition comprising a polymer-protein conjugate wherein a polymer molecule is attached to a functional group on a protein, and wherein the functional group is not reactive with the polymer molecule in the native conformation of the protein.

In another embodiment, the instant invention comprises a composition comprising a cytotoxic agent-protein conjugate wherein a cytotoxic agent is attached to a functional group on a protein, and wherein the functional group is not reactive with the cytotoxic agent in the native conformation of the protein.

In some embodiments, the reactive polymer molecule or the cytotoxic agent is covalently bound to the functional group on the protein.

In some embodiments, the functional group on the protein is an amino acid selected from the group consisting of cysteine, tyrosine, lysine, histidine, and glutamine. In some embodiments, the functional group on the protein is a native amino acid of the protein. In some embodiments, the functional group on the protein is a ligand or a cofactor.

In some embodiments, the protein is selected from the group consisting of antibodies, antibody fragments, antibodies and antibody fragments engineered to introduce cysteine residues, gluten proteins, low density lipoproteins, apolipoprotein A-I variants, proteins and peptide mimetics that contain the CAAX motif, mucolytics and mucins. In some embodiments, the protein is selected from the group consisting of glucocerebrosidase (GCB), II-1RA, G-CSF, Interferon Beta, basic fibroblast growth factor (bFGF), acidic fibroblast growth factor (aFGF), hemoglobin, thioredoxin, calcium- and integrin-binding protein 1 (CIB1), beta-lactoglobulin B, beta-lactoglobulin AB, serum albumin, core 2 beta 1,6-N-acetylglucosaminyltransferase-M (C2GnT-M), core 2 beta 1,6-N-acetylglucosaminyltransferase-I (C2GnT-I), platelet-derived growth factor receptor-beta (PDGF-beta), adenine nucleotide translocase (ANT), p53 tumor suppressor protein, acid sphingomyelinase, desfuroylceftiofur (DFC), apolipoprotein B100 (apoB), apolipoprotein A-I hypoxia-inducible factor-1 alpha (HIF-1 alpha), von Willebrand factor (VWF), carboxypeptidase Y, cathepsin B, cathepsin C, skeletal muscle Ca²⁺ release channel/ryanodine receptor (RyR1), nuclear factor kappa B (NF-KB), AP-1, protein-disulfide isomerase (PDI), glycoprotein lb alpha (GP1b alpha), calcineurin (CaN), fibrillin-1, CD4, S100A3 ionotropic glutamate receptors, human inter-alpha-inhibitor heavy chain 1, alpha2-antiplasmin (alpha2AP), thrombospondin, gelsolin, creatine kinase, Factor VIII, phospholipase D (PLD), insulin receptor beta subunit, acetylcholinesterase, prochymosin, modified alpha 2-macroglobulin (alpha 2M), glutathione reductase (GR), complement component C2, complement component C3, complement component 4, complement Factor B, alpha-lactalbumin, beta-D-galactosidase, endoplasmic reticulum Ca²⁺-ATPase, RNase inhibitor lipocortin 1, proliferating cell nuclear antigen (PCNA), actin, acyl-CoA synthetase, 3-2trans-enoyl-CoA-isomerase precursor atrial natriuretic factor (ANF)-sensitive guanylate cyclase, Pz-peptidase, aldehyde dehydrogenase, NADPH-P-450 reductase, glyceraldehydes-3-phosphate dehydrogenase (GAPDH), 6-pyruvoyl tetrahydropterin synthetase, lutropin receptor, low molecular weight acid phosphatase, serum cholinesterase (BChE), adrenodoxin, hyaluronidase, carnitine acyltransferases, interleukin-2 (IL-2), phosphoglycerate kinase, insulin-degrading enzyme (IDE), cytochrome c1 heme subunit, S-protein, valyl-tRNA synthetase (VRS), alpha-amylase I, muscle AMP deaminase, lactate dehydrogenase, somatostatin-binding protein, t-PA, and chondroitinase glycoprotein. In preferred embodiments the protein is G-CSF or Il-1RA.

In some embodiments, the protein has not been denatured. In some embodiments, the protein has not been treated with a chaotropic agent. In some embodiments, the protein has not been treated with a chaotropic agent prior to derivatization.

In some embodiments, the polymer-protein conjugate retains biological activity and in some embodiments, the polymer-protein conjugate has a greater in vivo specific activity than the protein.

In some embodiments, the reactive polymer is a synthetic polymer, a natural polymer, or a pseudosynthetic polymer. In some embodiments, the synthetic polymer may be PEG, N-(2-hydroxypropyl)-methacrylamide copolymers (HPMA), poly(ethyleneimine) (PEI), poly(acroloylmorpholine) (PAcM), poly(vinylpyrrolidone) (PVP), polyamidoamines, divinylethermaleic anhydride/acid copolymer (DIVEMA), poly(styrene-co-maleic acid/anhydride) (SMA), and polyvinylalcohol (PVA); the pseudosynthetic polymer may be PGA, poly(L-lysine), poly(malic acid), poly(aspartamides), and poly((Nhydroxyethyl)-L-glutamine) (PHEG); and the natural polymer may be dextran, pullulan, mannan, dextrin, chitosans, hyaluronic acid, protein, polysaccharide, DNA, and polysialic acid. In a preferred embodiment, the reactive polymer is PEG.

In some embodiments, the reactive polymer molecule may comprise a thiol specific, an amine specific, a hydroxyl specific, or a histidine specific reactive group. In preferred embodiments, the reactive polymer molecule is selected from the group consisting of maleimide, vinylsulfone, iodoacetyl, orthopyridyl-disulfide, succinimidyl succinate, succinimidyl carbonate, p-nitrophenyl carbonate, benzotriazolyl carbonate, trichlorophenyl carbonate, carbonylimidazole tresylate, dichlorotriazine, and aldehyde.

In some embodiments, the cytotoxic agent is selected from the group consisting of maytansinoid compounds, taxane compounds, CC-1065 compounds, daunorubicin compounds, doxorubicin compounds, and analogues or derivatives thereof. In some embodiments, the cytotoxic agent-protein conjugate is biologically active.

### BRIEF DESCRIPTION OF THE DRAWINGS

FIG. 1 shows a non-reducing SDS-polyacrylamide gel illustrating pegylated forms of G-CSF produced in accordance with the present invention.

FIG. 2 shows a non-reducing SDS-polyacrylamide gel illustrating pegylated forms of Il-1RA produced in accordance with the present invention.

### DETAILED DESCRIPTION

The present invention describes methods for derivatization of a protein by linking a polymer molecule or a cytotoxic agent to the protein using elevated hydrostatic pressure without requiring the use of denaturing levels of chaotropes, changes in pH, changes in temperature, or genetic modification of the native primary sequence of a recombinant protein. In this invention, elevated hydrostatic pressure is used to reversibly perturb the native conformation of a protein such that a normally buried or otherwise unreactive functional group, such as an amino acid residue, or a ligand or cofactor associated with the protein, is exposed and available for derivatization by a polymer molecule or a cytotoxic agent. Upon depressurization, the protein returns to its native conformation. Residues, such as cysteine, tyrosine, lysine, glutamine, or histidine may be targets for such site-specific derivatization. Methods of the present invention address many limitations of current technology by producing derivatized proteins without significant heterogeneity and reductions in specific activity. In addition, derivatized proteins made by the present invention do not require introduction of non-native amino acids and therefore have reduced risk of generating an immune response. Moreover, methods of the present invention allow for production of derivatized proteins with low ratios of polymer or cytotoxic agent to protein.

In one embodiment, the present invention includes a method for derivatization of a protein. This method includes applying hydrostatic pressure to the protein to increase reactivity of a functional group on the protein. The method also includes contacting the functional group on the protein with a reactive polymer molecule to form a polymer-protein conjugate. The method also includes depressurizing the polymer-protein conjugate.

Typically, the methods of the invention described herein are applied to solutions or mixtures where the total protein concentration is in the range of from about 0.001 mg/ml to about 500 mg/ml, from about 0.1 mg/ml to about 25 mg/ml or from about 1 mg/ml to about 10 mg/ml.

As is understood in the art, the term hydrostatic pressure means the pressure at a point in a fluid at rest due to the weight of the fluid above it. The method described herein involves raising the pressure above atmospheric pressure. Atmospheric pressure is approximately 15 pounds per square inch (psi) or 1 bar. In methods of the current invention, pressure may be generated using techniques and equipment known in the art for creating hydrostatic pressure. For example, hydraulic intensifier equipment may be used to create hydrostatic pressure on proteins. Proteins may be pressurized over time to a final desired pressure to reduce or avoid pressurization-induced heating. For example, proteins may be pressurized from atmospheric pressure to a final desired pressure over a time period of from about 10 minutes to about 48 hours, from about 60 minutes to about 24 hours, or from about 2 hours to about 16 hours.

Hydrostatic pressure has been shown to be an effective refolding tool, enabling refolding at relatively high concentration and with high yield. Such methods of refolding proteins using elevated hydrostatic pressure on solutions of proteins in order to disaggregate, unfold, and properly refold proteins are described in U.S. Pat. No. 6,489,450, U.S. Pat. No. 7,064,192, U.S. Patent Application Publication No. 2004/0038333, and International Patent Application WO 02/062827, each of which is incorporated by reference herein in their entirety.

Certain devices have also been developed which are particularly suitable for refolding of proteins under elevated pressure; see International Patent Application Publication No. WO 07/062174, which is incorporated by reference herein in its entirety.

In one embodiment, the instant method comprises applying hydrostatic pressure to a protein to increase reactivity of a functional group on the protein. Reference to increasing the reactivity of a functional group, which groups are described below, refers to the functional group reacting at a greater rate with another molecule, such as a reactive polymer molecule, when hydrostatic pressure is applied than when the protein is at atmospheric pressure. At atmospheric pressure the functional group may be non-reactive with other molecules or only less reactive than at higher pressures. For example, such functional groups may be groups that are normally buried, inaccessible or otherwise hindered from reaction with another molecule in the native conformation of the protein. Reference to the native conformation of a protein refers to the secondary, tertiary and quaternary structures of a protein when it is active. Thus, such functional groups, in the present context, are functional groups on the protein that in the native conformation of the protein have low chemical reactivity. In some embodiments, methods of the invention comprise applying hydrostatic pressure sufficient to disrupt the native conformation of a protein so that a functional group becomes exposed and accessible or more exposed and accessible to a polymer molecule, thus increasing its reactivity.

In some embodiments, applying a hydrostatic pressure to the protein to increase reactivity of a functional group of the protein comprises applying a pressure of from about 0.1 to about 25 kilobars, from about 0.5 to about 5 kilobars, or from about 1 to about 3 kilobars.

The method of the present invention further comprises contacting the functional group on the protein with a reactive polymer molecule to form a polymer-protein conjugate. In one embodiment, the reactive polymer molecule is present in the solution or reaction mixture containing the protein before the pressure is applied, but is not able to react with the functional group at atmospheric pressure. Upon application of pressure, the functional group becomes more reactive and when it is in the presence of the reactive polymer molecule, the reactive polymer molecule and the functional group react to form a polymer-protein conjugate. In another embodiment, hydrostatic pressure is applied to the protein without the reactive polymer being present and then after the protein is under pressure, the reactive polymer molecule is added to the protein solution or reaction mixture to form a polymer-protein conjugate. In some embodiments the protein solution or reaction mixture may comprise additional reagents, such as one or more catalysts to catalyze the reaction between the reactive polymer molecule and the functional group, buffers to maintain the pH in a range that allows the reactive polymer molecule and the functional group to react with each other to form the polymer-protein conjugate, and reducing agents such as DTT and TECP. Processes of the invention may be carried out any pH. Preferred pH for the processes of the invention ranges from about 4.0 to about 11.0, about 5.0 to pH about 10.0 and about 6.0 to about 9.0.

The method of the present invention further comprises depressurizing the protein-polymer conjugate. In one embodiment, after depressurization the derivatized protein is brought to atmospheric pressure. The step of depressurizing may be conducted at a suitable rate to produce proteins having native function. For example, the depressurization rate may be from about 1 bar per minute to about 20 bar per minute, from about 5 bar per minute to about 15 bar per minute, or from about 8 bar per minute to about 12 bar per minute.

The method may further comprise the step of recovering the polymer-protein conjugate after the step of depressurizing. The polymer-protein conjugates may be recovered by methods conventionally used for recovery of proteins. The polymer-protein conjugates may be stored as a solution in a suitable storage buffer, or may be lyophilized and stored in dry form.

The functional group on the protein may be an amino acid residue in the protein, a ligand associated with the protein, or a cofactor associated with the protein. In preferred embodiments of the present invention where the functional group on the protein is an amino acid, the functional group may be a cysteine, tyrosine, lysine, histidine, or glutamine. Such functional groups may be functional groups that naturally occur in a protein or they may be introduced to the protein, such as by genetic engineering techniques. In embodiments where the functional group on the protein is a ligand or cofactor, the functional group may be selected from the group consisting of nucleotide-containing ligands, heme-like moieties, metals, biotins, lipids, carbohydrates, peptides, enzyme substrates, catalysts or inhibitors. In some embodiments, the ligand or cofactor may be a non-native small molecule that is an analogue of a naturally occurring ligand. Examples of protein-ligand complexes include, without limitation, kinase-ATP complexes.

Methods of the present invention may be used to derivatize any protein, including antibodies, antibody fragments, gluten proteins, low density lipoproteins, apolipoprotein A-I variants, proteins and peptide mimetics that contain the CAAX motif, mucolytics and mucins. Specific examples include but are not limited to glucocerebrosidase (GCB), Il-1RA, G-CSF, Interferon Beta, basic fibroblast growth factor (bFGF), acidic fibroblast growth factor (aFGF), hemoglobin, thioredoxin, calcium- and integrin-binding protein 1 (CIB1), beta-lactoglobulin B, beta-lactoglobulin AB, serum albumin, core 2 beta 1,6-N-acetylglucosaminyltransferase-M (C2GnT-M), core 2 beta 1,6-N-acetylglucosaminyltransferase-I (C2GnT-I), platelet-derived growth factor receptor-beta (PDGF-beta), adenine nucleotide translocase (ANT), p53 tumor suppressor protein, acid sphingomyelinase, desfuroylceftiofur (DFC), apolipoprotein B100 (apoB), apolipoprotein A-I hypoxia-inducible factor-1 alpha (HIF-1 alpha), von Willebrand factor (VWF), carboxypeptidase Y, cathepsin B, cathepsin C, skeletal muscle Ca²⁺ release channel/ryanodine receptor (RyR1), nuclear factor kappa B (NF-KB), AP-1, protein-disulfide isomerase (PDI), glycoprotein lb alpha (GP1b alpha), calcineurin (CaN), fibrillin-1, CD4, S100A3 ionotropic glutamate receptors, human inter-alpha-inhibitor heavy chain 1, alpha2-antiplasmin (alpha2AP), thrombospondin, gelsolin, creatine kinase, Factor VIII, phospholipase D (PLD), insulin receptor beta subunit, acetylcholinesterase, prochymosin, modified alpha 2-macroglobulin (alpha 2M), glutathione reductase (GR), complement component C2, complement component C3, complement component 4, complement Factor B, alpha-lactalbumin , beta-D-galactosidase, endoplasmic reticulum Ca²⁺-ATPase, RNase inhibitor lipocortin 1, proliferating cell nuclear antigen (PCNA), actin, acyl-CoA synthetase, 3-2trans-enoyl-CoA-isomerase precursor atrial natriuretic factor (ANF)-sensitive guanylate cyclase, Pz-peptidase, aldehyde dehydrogenase, NADPH-P-450 reductase, glyceraldehydes-3-phosphate dehydrogenase (GAPDH), 6-pyruvoyl tetrahydropterin synthetase, lutropin receptor, low molecular weight acid phosphatase, serum cholinesterase (BChE), adrenodoxin, hyaluronidase, carnitine acyltransferases, interleukin-2 (IL-2), phosphoglycerate kinase, insulin-degrading enzyme (IDE), cytochrome cl heme subunit, S-protein, valyl-tRNA synthetase (VRS), alpha-amylase I, muscle AMP deaminase, lactate dehydrogenase, somatostatin-binding protein, t-PA, and chondroitinase glycoprotein. Suitable proteins also include any of the foregoing proteins or classes of proteins that have been modified, such as by deletions, substitutions or additions of amino acids, including without limitation, the introduction of functional groups.

Denatured as applied to a protein in the present context, means that native secondary and tertiary structure is disrupted, including cases in which the protein is denatured to an extent that it is no longer biologically active. Denaturation, in some cases, may allow exposure of amino acid residues that are buried or not reactive due to their inaccessibility to other reactive molecules in the native conformation of the protein. As is understood in the art, denaturation of a protein may be effected by treating the protein with a chaotropic agent. A chaotropic agent is a compound, including, without limitation, guanidine hydrochloride (guanidinium hydrochloride, GdmHCl), sodium thiocyanate, urea and/or a detergent which disrupts the noncovalent intra-molecular bonds within the protein, permitting the amino acid chain to assume a substantially random or non-native conformation. The methods of the present invention may be applied to proteins that are not denatured or not treated with chaotropic agents, prior to derivatization or during derivatization. However, the invention does not exclude embodiments where a chaotropic agent is either included in and/or added to the protein mixture. In such embodiments the concentration of chaotropic agent is limited to that which permits retention of biological activity of the protein in its native form.

As understood in the art, a polymer is a substance composed of repeating structural units, or monomers, connected by covalent chemical bonds. In the present methods, the reactive polymer molecule may be a synthetic polymer, a natural polymer, or a pseudosynthetic polymer. Examples of synthetic polymers include, without limitation, polyethyelene glycol (PEG), N-(2-hydroxypropyl)-methacrylamide copolymers (HPMA), poly(ethyleneimine) (PEI), poly(acroloylmorpholine) (PAcM), poly(vinylpyrrolidone) (PVP), polyamidoamines, divinylethermaleic anhydride/acid copolymer (DIVEMA), poly(styrene-co-maleic acid/anhydride) (SMA), and polyvinylalcohol (PVA) Examples of pseudosynthetic polymer include, without limitation, polyglutamic acid (PGA), poly(L-lysine), poly(malic acid), poly(aspartamides), and poly((Nhydroxyethyl)-L-glutamine) (PHEG). Examples of natural polymers include, without limitation, dextran, pullulan, mannan, dextrin, chitosans, hyaluronic acid, protein, polysaccharide, DNA, and polysialic acid. The reactive polymer molecule may also be a complex polymer molecule that comprises more than one type of polymer moiety, for example a PEG moiety attached to a poly-lysine moiety.

In a preferred embodiment, the reactive polymer molecule is PEG. The term PEG or polyethylene glycol refers to a polymer of ethylene oxide molecules and includes polymer molecules of varying polymer lengths and molecular weights. For example, PEG molecules are currently commercially available over a wide range of molecular weights ranging from about 100 to about 50,000,000 Daltons. Furthermore, PEG molecules may have different geometries, and for example, may be linear or branched. The term PEG molecules may also refer to modified forms of PEG that are obtained, depending on the initiator used in the polymerization process, such as methoxy polyethylene glycol or mPEG. The term PEG or PEG molecules as used herein encompasses all forms of PEG molecules known in the art. In preferred embodiments the PEG molecule has a molecular weight ranging from about 1000 Daltons to about 500,000 Daltons, from about 3000 Daltons to about 250,000 Daltons, from about 5000 Daltons to about 100,000 Daltons, or from about 10,000 Daltons to about 50,000 Daltons, or from about 20,000 Daltons to about 40,000 Daltons.

In one embodiment, the reactive polymer molecule comprises a thiol specific reactive group. Examples of such polymers include but are not limited to maleimide, vinylsulfone, iodoacetyl, and orthopyridyl-disulfide. In another embodiment, the reactive polymer molecule comprises an amine specific reactive group. Examples of such polymer include, but are not limited to, succinimidyl succinate, succinimidyl carbonate, p-nitrophenyl carbonate, benzotriazolyl carbonate, trichlorophenyl carbonate, carbonylimidazole tresylate, and dichlorotriazine aldehyde. In another embodiment, the reactive polymer molecule comprises a hydroxyl specific reactive group. Examples of such polymers include, but are not limited to, succinimidyl succinate, benzotriazolyl carbonate, and dichlorotriazine. In another embodiment, the reactive polymer molecule comprises a histidine specific reactive group. Examples of such polymers include but are not limited to succinimidyl succinate, benzotriazolyl carbonate and dichlorotriazine.

In some embodiments, the protein polymer conjugate retains the biological activity of the protein. Biological activity of a protein as used herein, means at least about 10%, at least about 20%, at least about 30%, at least about 40%, at least about 50%, at least about 60%, at least about 70%, at least about 80%, at least about 90%, or at least about 95% of maximal known specific activity as measured in an assay that is generally accepted in the art to be correlated with the known or intended utility of the protein. For proteins intended for therapeutic use, the assay of choice may be one accepted by a regulatory agency to which data on safety and efficacy of the protein is submitted. A protein having greater than 10% of maximal known specific activity is “biologically active” for the purposes of the invention. In some embodiments the polymer-protein conjugate may have a greater in vivo specific activity than the protein.

In some embodiments the molar ratio of the reactive polymer molecule to protein in the reaction mixture is less than about 20. In other embodiments, the molar ratio of the reactive polymer molecule to protein is less than about 10. In still other embodiments, the molar ratio of the reactive polymer molecule to protein is less than about 5.

The processes of the invention may be carried out at any temperature between the freezing point of an aqueous medium (about 0° C.) containing the protein and the temperature at which biological activity of the protein is lost due to thermal denaturation. The upper limit will be somewhat different for each individual protein and will also be affected by the composition of the medium, pH, presence of stabilizing compounds and the like, as is known in the art. Preferred temperatures for carrying out the process of the invention are within the ranges of about 5° C., about 10° C., or about 20° C. below the temperature at which biological activity is lost and the temperature at which biological activity is lost. In another embodiment, temperatures for carrying out the process of the invention range from about 4° C. to about 37° C.

Another embodiment of the present invention includes a method for derivatization of a protein, which includes applying hydrostatic pressure to the protein of about 0.1 to about 25 kilobars. The method also includes contacting a functional group on the protein with a reactive polymer molecule to form a polymer-protein conjugate. The method also includes depressurizing the protein-polymer conjugate. In some embodiments, the hydrostatic pressure applied is sufficient to alter the native conformation of the protein. Preferred embodiments may include applying hydrostatic pressures to the protein of about 0.25 to about 5 kilobars, or about 1 to about 3 kilobars.

Another embodiment of the present invention includes a polymer-protein conjugate wherein a polymer molecule is attached to a functional group on a protein, and wherein the functional group is not reactive with the polymer molecule in the native conformation of the protein. It may be that the functional group is normally buried or inaccessible or otherwise hindered from reaction with the polymer molecule in the native conformation of the protein and therefore is not reactive with the polymer molecule in the native conformation of the protein. In some embodiments, the polymer molecule is covalently bound to the protein. The functional group may be an amino acid residue in the protein. In preferred embodiments, the functional group may be a cysteine, tyrosine, lysine, histidine, or glutamine. Some embodiments include polymer-protein conjugates that are prepared by the methods described in the present invention.

Another embodiment of the present invention includes a method for derivatization of a protein with a cytotoxic agent, which includes applying hydrostatic pressure to the protein to increase reactivity of a functional group on the protein. The method also includes contacting the functional group on the protein with a cytotoxic agent to form a cytotoxic agent-protein conjugate. The method also includes depressurizing the cytotoxic agent-protein conjugate. After depressurizing the protein may be brought to atmospheric pressure. The method may further comprise the step of recovering the cytotoxic agent-protein conjugate after the step of depressurizing the protein. As understood in the art a cytotoxic agent refers to a molecule that is toxic to living cells. Cytotoxic agents may include, without limitation, chemicals, drugs, peptides, hormones, antibodies or antibody fragments. In preferred embodiments, cytotoxic agents may include maytansinoid compounds, taxane compounds, CC-1065 compounds, daunorubicin compounds, doxorubicin compounds, and analogues or derivatives thereof.

In one embodiment the molar ratio of the cytotoxic agent to protein is less than about 20. In other embodiments the molar ratio of the cytotoxic agent to protein is less than about 10, or less than about 5. In some embodiments the cytotoxic agent-protein conjugate is biologically active. As used herein, a biologically active cytotoxic agent-protein means that the cytotoxic agent-protein conjugate has some effect on or interacts with living cells; such effect or interaction may be beneficial or adverse to the cells. In further embodiments, the cytotoxic agent may comprise a thiol specific reactive group, an amine specific reactive group, a hydroxyl specific reactive group, or a histidine specific reactive group.

Another embodiment of the present invention includes a method for derivatization of a protein with a cytotoxic agent, which includes applying to the protein a hydrostatic pressure of about 0.1 to about 25 kilobars. The method also includes contacting the functional group on the protein with a cytotoxic agent to form a cytotoxic agent-protein conjugate. The method also includes depressurizing the cytotoxic agent-protein conjugate. In some embodiments the hydrostatic pressure applied is sufficient to alter the native conformation of the protein. Preferred embodiments include applying hydrostatic pressures to the protein of about 0.25 to about 5 kilobars, or about 1 to about 3 kilobars.

Another embodiment of the present invention includes a cytotoxic agent-protein conjugate wherein a cytotoxic agent is attached to a functional group on a protein, and wherein the functional group is not reactive with the cytotoxic agent in the native conformation of the protein. It may be that the functional group is normally buried or inaccessible or otherwise hindered from reaction with the cytotoxic agent in the native conformation of the protein and therefore is not reactive with the cytotoxic agent in the native conformation of the protein. In some embodiments the cytotoxic agent is covalently bound to the protein. The functional group may be an amino acid residue in the protein. In preferred embodiments, the functional group may be a cysteine, tyrosine, lysine, histidine, or glutamine.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

### EXAMPLES

### Example 1

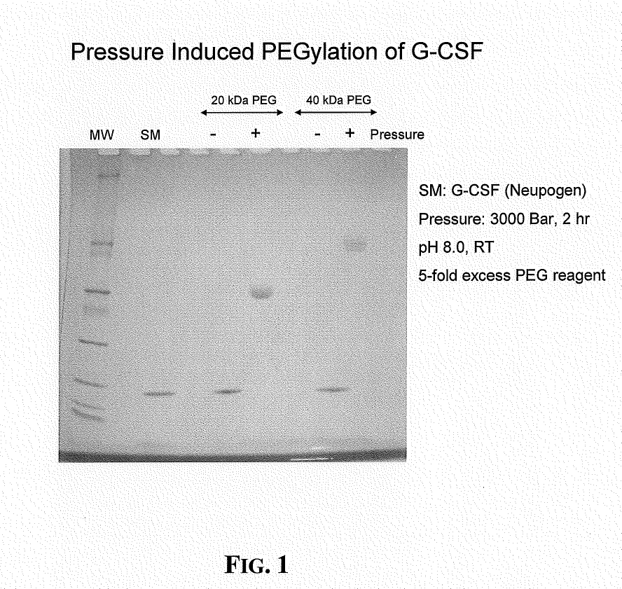

This example illustrates production of pegylated G-CSF molecules prepared in accordance with the present invention. G-CSF (NEUPOGEN®, Amgen Inc., Thousand Oaks, Calif.) was diluted with 100 mM Tris pH 8 to a final concentration of 100 μg/ml. A 5× fold excess of 20 kDa or branched 40 kDa maleimide (Nippon Oil and Fats Co., Ltd., Tokyo, Japan) was added to two ml aliquots of the diluted protein. One ml of each pegylation reaction mixture was loaded into a caisson and subjected to high hydrostatic pressure (3 kilobars) for 2 hours at room temperature. Pressure was generated using high-pressure nitrogen (400 bar) connected to a 10-fold hydraulic intensifier equipment (High Pressure Equipment Company, Erie, Pa.). The remainder of the pegylation reaction mixture was allowed to sit at atmospheric pressure. After depressurizaton, the protein samples were analyzed by non-reducing SDS-PAGE analysis (10-20% polyacrylamide gels, NOVEX®, San Diego, Calif.) using a coomassie stain for detection. As can be seen in FIG. 1, at atmospheric pressure, the free cysteine at position 17 remained buried and thus was non-reactive. Upon exposure to hydrostatic pressure of 3 kilobars, no detectable unmodified starting material and a single pegylated form of G-CSF for both the 20 and 40 kDa PEG reagents was observed.

### EXAMPLE 2

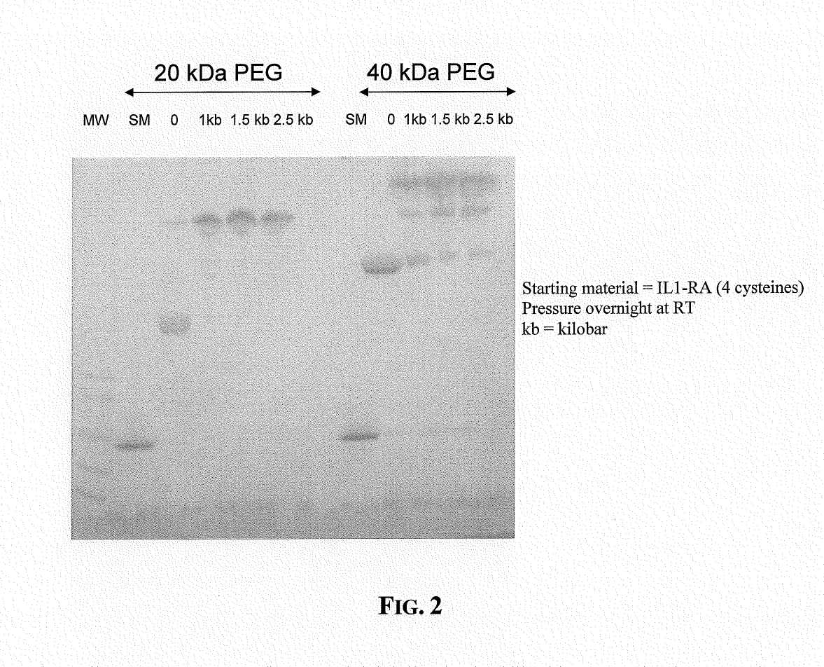

This example illustrates production of pegylated Il-1RA molecules prepared in accordance with the present invention. Il-1RA (KINERET® , Amgen, Inc, Thousand Oaks, Calif.) was diluted to a final concentration of 1 mg/ml with 100 mM Tris, pH 8. A 5× fold excess of 20 kDa or branched 40 kDa Maleimide PEG (NOF) was added to 2 ml aliquots. One ml of each pegylation reaction mixture was loaded into individual caissons and subjected to hydrostatic pressures ranging from 1 kilobar to 2.5 kilobars for 16 hour at room temperature. The remaining 1 ml of the pegylation reaction mixture was allowed to sit at room temperature for the same amount of time. After depressurizaton, the protein samples were analyzed by reducing SDS-PAGE analysis (10-20% polyacrylamide gels, NOVEX®, San Diego, Calif.) using a coomassie stain for detection. The results are shown in FIG. 2. At atmospheric pressure, only one cysteine reacted with the PEG reagent. At pressures ranging from 1 kilobar to 2.5 kilobars, additional cysteines were able to react with both the 20 kDa and 40 kDa PEG reagents.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method for derivatization of a protein comprising the steps of:
a) applying hydrostatic pressure to the protein to increase reactivity of a functional group on the protein;
b) contacting the functional group on the protein with a reactive polymer molecule to form a polymer-protein conjugate; and
c) depressurizing the polymer-protein conjugate.

2. The method of claim 1, further comprising the step of recovering the polymer-protein conjugate.

3. The method of claim 1, wherein the functional group on the protein is an amino acid selected from the group consisting of cysteine, tyrosine, lysine, histidine, and glutamine.

4. The method of claim 1, wherein the protein is selected from the group consisting of antibodies, antibody fragments, antibodies and antibody fragments engineered to introduce cysteine residues, gluten proteins, low density lipoproteins, apolipoprotein A-I variants, proteins and peptide mimetics that contain the CAAX motif, mucolytics and mucins.

5. The method of claim 1, wherein the protein is selected from the group consisting of glucocerebrosidase (GCB), II-1RA, G-CSF, Interferon Beta, basic fibroblast growth factor (bFGF), acidic fibroblast growth factor (aFGF), hemoglobin, thioredoxin, calcium- and integrin-binding protein 1 (CIB1), beta-lactoglobulin B, beta-lactoglobulin AB, serum albumin, core 2 beta 1,6-N-acetylglucosaminyltransferase-M (C2GnT-M), core 2 beta 1,6-N-acetylglucosaminyltransferase-I (C2GnT-I), platelet-derived growth factor receptor-beta (PDGF-beta), adenine nucleotide translocase (ANT), p53 tumor suppressor protein, acid sphingomyelinase, desfuroylceftiofur (DFC), apolipoprotein B 100 (apoB), apolipoprotein A-I hypoxia-inducible factor-1 alpha (HIF-1 alpha), von Willebrand factor (VWF), carboxypeptidase Y, cathepsin B, cathepsin C, skeletal muscle Ca²⁺ release channel/ryanodine receptor (RyR1), nuclear factor kappa B (NF-KB), AP-1, protein-disulfide isomerase (PDI), glycoprotein 1b alpha (GP1b alpha), calcineurin (CaN), fibrillin-1, CD4, S100A3 ionotropic glutamate receptors, human inter-alpha-inhibitor heavy chain 1, alpha2-antiplasmin (alpha2AP), thrombospondin, gelsolin, creatine kinase, Factor VIII, phospholipase D (PLD), insulin receptor beta subunit, acetylcholinesterase, prochymosin, modified alpha 2-macroglobulin (alpha 2M), glutathione reductase (GR), complement component C2, complement component C3, complement component 4, complement Factor B, alpha-lactalbumin, beta-D-galactosidase, endoplasmic reticulum Ca²⁺-ATPase, RNase inhibitor lipocortin 1, proliferating cell nuclear antigen (PCNA), actin, acyl-CoA synthetase, 3-2trans-enoyl-CoA-isomerase precursor atrial natriuretic factor (ANF)-sensitive guanylate cyclase, Pz-peptidase, aldehyde dehydrogenase, NADPH-P-450 reductase, glyceraldehydes-3-phosphate dehydrogenase (GAPDH), 6-pyruvoyl tetrahydropterin synthetase, lutropin receptor, low molecular weight acid phosphatase, serum cholinesterase (BChE), adrenodoxin, hyaluronidase, carnitine acyltransferases, interleukin-2 (IL-2), phosphoglycerate kinase, insulin-degrading enzyme (IDE), cytochrome c1 heme subunit, S-protein, valyl-tRNA synthetase (VRS), alpha-amylase I, muscle AMP deaminase, lactate dehydrogenase, somatostatin-binding protein, t-PA, and chondroitinase glycoprotein.

6. The method of claim 1, wherein the protein is G-CSF, or Il-1RA, or Interferon Beta.

7. The method of claim 1, wherein the protein has not been denatured.

8. The method of claim 1, wherein the protein has not been treated with a chaotropic agent.

9. The method of claim 1, where in the protein has not been treated with a chaotropic agent prior to derivatization.

10. The method of claim 1, wherein the functional group on the protein is a native amino acid of the protein.

11. The method of claim 1, wherein molar ratio of the reactive polymer molecule to protein is less than about 20.

12-13. (canceled)

14. The methods of claims 11, wherein the reactive polymer is PEG.

15. The method of claim 1, wherein the polymer-protein conjugate retains biological activity.

16. The method of claim 1, wherein the reactive polymer molecule is present with the protein during step a.

17. The method of claim 1, wherein after depressurization the derivatized protein is brought to atmospheric pressure.

18. The method of claim 1, wherein the reactive polymer is a synthetic polymer,
a natural polymer, or a pseudosynthetic polymer.

19. The method of claim 18, wherein the synthetic polymer is selected from the group consisting of PEG, N-(2-hydroxypropyl)-methacrylamide copolymers (HPMA), poly(ethyleneimine) (PEI), poly(acroloylmorpholine) (PAcM), poly(vinylpyrrolidone) (PVP), polyamidoamines, divinylethermaleic anhydride/acid copolymer (DIVEMA), poly(styrene-co-maleic acid/anhydride) (SMA), and polyvinylalcohol (PVA).

20. The method of claim 18, wherein the pseudosynthetic polymer is selected from the group consisting of PGA, poly(L-lysine), poly(malic acid), poly(aspartamides), and poly((Nhydroxyethyl)-L-glutamine) (PHEG).

21. The method of claim 18, wherein the natural polymer is selected from the group consisting of dextran, pullulan, mannan, dextrin, chitosans, hyaluronic acid, protein, polysaccharide, DNA, and polysialic acid.

22. The method of claim 1, wherein the polymer-protein conjugate has a greater in vivo specific activity than the protein.

23. The method of claim 1, wherein the reactive polymer molecule comprises a thiol specific reactive group.

24. The method of claim 23, wherein the reactive polymer molecule is selected from the group consisting of maleimide, vinylsulfone, iodoacetyl, and orthopyridyl-disulfide.

25. The method of claim 1, wherein the reactive polymer molecule comprises an amine specific reactive group.

26. The method of claim 25, wherein the reactive polymer molecule is selected from the group consisting of succinimidyl succinate, succinimidyl carbonate, p-nitrophenyl carbonate, benzotriazolyl carbonate, trichlorophenyl carbonate, carbonylimidazole tresylate, dichlorotriazine, and aldehyde.

27. The method of claim 1, wherein the reactive polymer molecule comprises a hydroxyl specific reactive group.

28. The method of claim 27, wherein the reactive polymer molecule is selected from the group consisting of succinimidyl succinate, benzotriazolyl carbonate, and dichlorotriazine.

29. The method of claim 1, wherein the reactive polymer molecule comprises a histidine specific reactive group.

30. The method of claim 29, wherein the reactive polymer molecule is selected from the group consisting of succinimidyl succinate, benzotriazolyl carbonate, and dichlorotriazine.

31. A method for derivatization of a protein comprising the steps of:
a) applying to the protein a hydrostatic pressure of about 0.1 to about 25 kilobars;
b) contacting a functional group on the protein with a reactive polymer molecule to form a polymer-protein conjugate;
c) and depressurizing the protein-polymer conjugate.

32. The method of claim 31, further comprising the step of recovering the derivatized protein.

33. The method of claim 31, wherein the hydrostatic pressure applied to the protein is sufficient to alter native conformation of the protein.

34-63. (canceled)

64. A method for derivatization of a protein comprising the steps of:
a) applying hydrostatic pressure to the protein to increase reactivity of a functional group on the protein;
b) contacting the functional group on the protein with a cytotoxic agent to form a cytotoxic agent-protein conjugate; and
c) depressurizing the cytotoxic agent-protein conjugate.

65-108. (canceled)

109. A composition comprising a polymer-protein conjugate or a cytotoxic agent-protein conjugate wherein a polymer molecule or a cytotoxic agent is attached to a functional group on a protein, and wherein the functional group is not reactive with the polymer molecule or the cytotoxic agent in the native conformation of the protein.

110-157. (canceled)
